(19) United States
(12) Patent Application Publication
(10) Pub. No.: US 2026/0192464 A1
(43) Pub. Date: Jul. 9, 2026

(54) System and Method for Integrating Real-Time Mobility Data with Local Assistance Robots for Enhanced Patient Care

(71) Applicant: Stanley G. Van Meter, II, Lake Mary, FL (US)

(72) Inventor: Stanley G. Van Meter, II, Lake Mary, FL (US)

(21) Appl. No.: 19/426,905

(22) Filed: Dec. 19, 2025

Related U.S. Application Data
(60) Provisional application No. 63/736,687, filed on Dec. 20, 2024.

(51) Int. Cl. B25J 11/00; A61H 3/00; B25J 9/16; G16H 50/30

(52) U.S. Cl. CPC B25J 11/009; A61H 3/00; B25J 9/163; G16H 50/30; A61H 2201/1659; A61H 2201/5007; A61H 2201/5084; A61H 2230/625

(57) ABSTRACT

A system and method are provided for integrating real-time mobility and balance data with local assistance robots to enhance patient safety and care. The system collects mobility and balance data from a health-monitoring app and transmits it to a local assistance robot. Based on predefined thresholds, the robot dynamically shifts into a guardian mode, proactively assisting the user with verbal cues, physical stabilization, or emergency interventions. The robot employs machine learning to adapt to individual user behaviors, improving the accuracy of interventions over time. In guardian mode, the robot tracks mobility metrics in real time and provides personalized support, including fall prevention, assistance with walking, and emergency alerts. This integration ensures seamless communication between monitoring systems and robotic assistants, enabling immediate and autonomous responses to potential mobility risks. The invention offers improved safety, reduced fall risks, and personalized care for individuals with chronic conditions, mobility impairments, or advanced age.

### FEDERALLY SPONSORED RESEARCH

Not Applicable

### SEQUENCE LISTING OR PROGRAM

Not Applicable

### TECHNICAL FIELD OF THE INVENTION

The present invention relates to systems and methods for integrating real-time mobility data. Specifically, the present invention pertains to systems and methods for integrating real-time mobility data that use local assistant robots for enhanced patient care. The present invention enables robots to shift into guardian mode, offering real-time support and interventions based on a patient's mobility status, providing enhanced safety and care in a home or clinical setting.

### BACKGROUND OF THE INVENTION

This invention relates to systems and methods for enhancing patient safety and mobility assistance through the integration of real-time balance and mobility data with local assistance robots. Patients, particularly those with mobility impairments, chronic conditions, or advanced age, face significant risks associated with falls and sudden declines in balance or gait stability. Addressing these risks in a timely manner is critical to maintaining safety, reducing injuries, and ensuring a high quality of life for individuals who require mobility support.

Current health-monitoring systems are capable of tracking balance and mobility metrics, often using smartphone-based sensors or wearable devices. While these systems provide valuable insights into a patient's condition, they lack the ability to physically assist the user when potential risks are detected. At the same time, local assistance robots have advanced in their ability to offer mobility support and caregiving functions but require commands or prompts to engage with users, limiting their effectiveness in real-time risk scenarios.

This invention bridges the gap between real-time data monitoring and physical intervention by enabling local assistance robots to respond autonomously to mobility and balance data. By integrating mobility data from a health-monitoring system with the robot's control mechanisms, the system facilitates dynamic, proactive interventions. These interventions range from verbal cues to physical stabilization, allowing robots to act as guardians for individuals at risk of falls or mobility-related incidents. The invention provides a comprehensive solution for enhancing safety and care, leveraging advanced data analytics and robotics to deliver real-time, personalized assistance.

### SUMMARY OF THE INVENTION

The present invention provides a system and method for integrating real-time mobility and balance data with local assistance robots to enhance patient safety and care. By leveraging advanced data collection from a health-monitoring application and integrating it with a robotic system, the invention allows for proactive interventions in response to detected mobility risks. The health-monitoring application collects real-time data, such as gait speed, stride stability, and postural sway, and transmits this information to the robot's control system using secure communication protocols.

The system employs predefined thresholds and machine learning algorithms to analyze mobility data and detect potential risks. When a risk is identified, the robot shifts into a “guardian mode,” where it dynamically adjusts its behavior to provide interventions tailored to the user's needs. Interventions include verbal cues for mild risks, physical stabilization for moderate risks, and emergency support for imminent fall scenarios. Over time, the system personalizes its responses by learning from historical mobility data, adapting its thresholds and interventions to the user's specific patterns.

This invention addresses critical gaps in patient mobility care by providing continuous monitoring, real-time intervention, and personalized support. The integration of predictive analytics and robotic assistance ensures enhanced safety and reduced fall risks for individuals with mobility challenges, such as seniors or patients with chronic conditions.

### BRIEF DESCRIPTION OF THE DRAWINGS

No drawings are submitted with the present provisional application. The invention is fully described and enabled through the written specification, which defines the structural and functional relationships of the modular sampling insert architecture independent of any particular geometric depiction. Any prior references to figures in earlier drafts were illustrative only and are not necessary for understanding or practicing the disclosed invention.

### DETAILED DESCRIPTION OF THE INVENTION

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

The invention provides a comprehensive system and method for integrating real-time mobility and balance data with local assistance robots to enhance patient safety and care. By combining health-monitoring technologies with robotic assistance, this system enables dynamic, proactive interventions to address mobility risks and prevent injuries. The system collects mobility and balance data through a health-monitoring app, such as Balance EQ, which uses sensors embedded in smartphones or wearable devices to measure metrics like gait speed, stride stability, and postural sway. This data is transmitted wirelessly to the control unit of a local assistance robot, such as Tesla Optimus, using secure communication protocols. The robot's control system processes the incoming data to assess the user's mobility status and determine whether intervention is necessary.

In standard operation, the robot performs routine functions and monitors the user's mobility without active intervention unless a risk is detected. When the mobility data indicates a potential risk, such as a sudden change in gait speed or an increase in instability, the robot shifts into a heightened state of operation referred to as “guardian mode.” In this mode, the robot becomes more attentive, staying closer to the user and preparing to provide support. The robot is capable of a range of interventions tailored to the severity of the detected risk. For mild risks, such as a decrease in walking speed, the robot issues verbal cues, such as reminders to slow down or hold onto a stable surface. For moderate risks, the robot offers physical stabilization, such as providing a supportive grip or steadying the user during walking. In high-risk scenarios, such as an imminent fall, the robot enters emergency mode, physically preventing the fall or alerting caregivers to provide further assistance.

The system incorporates machine learning algorithms to enhance its ability to assist users effectively. By analyzing historical mobility data, the robot learns individual patterns of movement and behavior, enabling it to anticipate risks and adjust its interventions accordingly. Over time, the system personalizes its responses, refining its guardian mode to align with the user's unique needs and conditions. For instance, a user with a consistently slower gait may experience fewer verbal prompts, while a user prone to stumbling may trigger more frequent interventions. This adaptive capability ensures that the system delivers precise and effective assistance, improving both safety and user satisfaction.

The integration of real-time data collection, risk detection, and tailored interventions forms the core of this invention's workflow. Mobility data is continuously transmitted from the health-monitoring app to the robot, where it is analyzed against predefined thresholds to identify potential risks. When a risk is detected, the robot's control unit determines the most appropriate course of action, ranging from verbal assistance to physical intervention. Once the intervention is complete, the system evaluates the outcome, incorporating feedback into its algorithms to improve future performance.

This invention has numerous applications, including fall prevention for seniors, assistance for patients with chronic mobility conditions, and emergency response for unexpected incidents. For example, a senior at risk of falling can rely on the robot for immediate stabilization, while a patient with Parkinson's disease experiencing gait freezing can receive both verbal cues and physical support to regain movement. In more severe cases, such as an imminent fall, the robot's emergency mode ensures the user's safety by providing physical support and alerting caregivers or emergency services.

By integrating real-time mobility data with the proactive capabilities of local assistance robots, this invention addresses critical gaps in patient safety and mobility care. It provides continuous monitoring and immediate intervention, reducing the risk of falls and improving the quality of life for individuals with mobility challenges. The system's ability to learn and adapt to individual behaviors further enhances its effectiveness, making it an essential tool for healthcare providers, caregivers, and users.

The system is set to run on a computing device or mobile electronic device. A computing device or mobile electronic device on which the present invention can run would be comprised of a CPU, Hard Disk Drive, Keyboard, Monitor, CPU Main Memory and a portion of main memory where the system resides and executes. Any general-purpose computer, smartphone, or other mobile electronic device with an appropriate amount of storage space is suitable for this purpose. Computer and mobile electronic devices like these are well known in the art and are not pertinent to the invention. The system can also be written in a number of different languages and run on a number of different operating systems and platforms.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

Furthermore, other areas of art may benefit from this method and adjustments to the design are anticipated. Thus, the scope of the invention should be determined by the appended claims and their legal equivalents, rather than by the examples given.

1. A non-transitory computer-readable medium storing instructions that, when executed by a processor, enable a system to integrate real-time mobility and balance data with a local assistance robot, the system comprising:
a health-monitoring application configured to collect mobility and balance data from a user;
a communication module configured to transmit the collected data to the local assistance robot;
a control system within the local assistance robot configured to analyze the data and detect a mobility risk based on predefined thresholds; and
a set of algorithms that enable the robot to perform interventions in response to the detected mobility risk.

2. The system of claim 1, further comprising:
a health-monitoring application communicatively coupled to an accelerometer;
a robot configured to receive mobility data from the health-monitoring application; and
a machine learning model within the robot configured to adaptively determine user-specific risk thresholds and customize interventions based on historical mobility data.

3. The system of claim 1, further comprising storing instructions that enable a system to dynamically shift a local assistance robot into a guardian mode, wherein the system comprises:
a mobility monitoring application configured to generate real-time balance data;
a robot control unit communicatively coupled to the application; and
a decision-making engine within the robot configured to adjust the robot's behavior based on the balance data.

4. The system of claim 1, wherein the storing instructions further comprising:
real-time communication between a health-monitoring application and a local assistance robot;
the local assistance robot to detect potential falls; and
automated interventions including verbal assistance, physical stabilization, and emergency alerts.

5. The system of claim 1, further comprising storing instructions for
continuously monitoring mobility data;
calculating the probability of a fall based on detected anomalies in the data; and
triggering a physical support intervention from a local assistance robot.

6. The system of claim 1, further comprising storing instructions for:
a mobile device configured to transmit real-time mobility metrics;
a local assistance robot equipped with sensors for providing physical assistance; and
a processor configured to integrate user mobility patterns into a fall prediction model.

7. The system of claim 1, further comprising storing instructions for:
an alert system within a local assistance robot that provides real-time feedback to a user; and
a mobility data analytics system for identifying potential fall risks based on accelerometer readings.

8. The system of claim 1, further comprising:
storing instructions that enable a system to:
receive and process real-time gait and balance data;
compare the data against user-specific risk thresholds; and
dynamically adjust the robot's proximity and assistance level.

9. The system of claim 1, further comprising:
storing instructions that enable a system to:
predict falls using machine learning algorithms trained on historical mobility data; and
initiate an emergency protocol, including stabilizing the user and notifying caregivers.

10. The system of claim 1, further comprising:
storing instructions to enable a local assistance robot to:
monitor a user's mobility patterns;
identify deviations indicating risk; and
adjust interventions dynamically to reduce false positives.

11. A method for integrating mobility data with a local assistance robot, comprising:
collecting real-time balance data from a health-monitoring application;
transmitting the data to the robot;
analyzing the data for mobility risks using a control system within the robot; and
initiating an intervention based on detected risks.

12. The method of claim 11, further comprising operating a local assistance robot in a guardian mode, comprising:
detecting a mobility risk using real-time gait data;
shifting the robot into a heightened state of vigilance; and
assisting the user with verbal cues or physical stabilization.

13. The method of claim 11, further comprising a method for fall prevention using real-time mobility data, comprising:
continuously monitoring balance metrics;
comparing the metrics to predefined risk thresholds; and
triggering a robot to intervene when a threshold is exceeded.

14. The method of claim 11, further comprising a method for personalizing robot interventions, comprising:
collecting historical balance data;
training a machine learning model to recognize user-specific mobility patterns; and
tailoring interventions based on the model's predictions.

15. The method of claim 11, further comprising a method for emergency fall intervention, comprising:
detecting a sudden instability in a user's gait;
initiating a physical stabilization response by a robot; and
notifying caregivers through a network-enabled device.

16. The method of claim 11, further comprising a method for dynamic monitoring of user mobility, comprising:
analyzing accelerometer data to detect anomalies in stride or gait;
activating a local assistance robot to provide support; and
logging the event for future analysis.

17. The method of claim 11, further comprising a method for integrating real-time mobility data with robotic assistance, comprising:
transmitting gait data from a mobile device to a local robot;
evaluating the data to identify potential risks; and
adjusting the robot's behavior to provide optimal assistance.

18. The method of claim 11, further comprising a method for preventing falls using a local assistance robot, comprising:
monitoring a user's walking patterns;
detecting deviations indicating instability; and
physically supporting the user through the robot's mechanisms.

19. The method of claim 11, further comprising a method for real-time intervention using balance data, comprising:
collecting real-time balance metrics from a mobile device;
identifying fall risks using predictive algorithms; and
initiating an intervention by a local assistance robot.

20. The method of claim 11, further comprising a method for adaptive robotic assistance, comprising:
processing mobility data using a machine learning model;
dynamically updating risk thresholds; and
adjusting the robot's proximity and support level to match the user's needs.
